(19) United States
(12) Patent Application Publication
(10) Pub. No.: US 2025/0268829 A1
(43) Pub. Date: Aug. 28, 2025

(54) INHALED ANTIBACTERIAL FORMULATIONS FOR TREATING LUNG INFECTIONS

(71) Applicant: Board of Regents, The University of Texas System, Austin, TX (US)

(72) Inventors: Varsha V. Nair, Austin, TX (US); Hugh Smyth, Austin, TX (US)

(73) Assignee: Board of Regents, The University of Texas System, Austin, TX (US)

(21) Appl. No.: 18/855,984

(22) PCT Filed: Apr. 21, 2023
(86) PCT No.: PCT/US2023/019352

§ 371 Date: Oct. 10, 2024

Related U.S. Application Data
(60) Provisional application No. 63/333,626, filed on Apr. 22, 2022.

(51) Int. Cl. A61K 9/14; A61K 9/00; A61K 31/65; A61P 31/04

(52) U.S. Cl. CPC A61K 9/14; A61K 9/0073; A61K 31/65; A61P 31/04

(57) ABSTRACT

Described herein is a composition comprising a micronized tigecycline having a Dv90 mean particle size of less than 7 μm, wherein the composition is a powder, wherein the composition is carrier free, and wherein the composition is free of any stabilizing excipients. Also, described herein is a method of treating a pulmonary infection in a subject, comprising administering to a subject a composition of micronized tigecycline. The composition can be administered by means of local delivery, pulmonary delivery, or inhaled delivery.

### CROSS REFERENCE TO RELATED APPLICATIONS

This application claims the benefit of and priority to U.S. Provisional Application No. 63/333,626, filed on Apr. 22, 2022, which is hereby incorporated by reference in its entirety.

### BACKGROUND

Traditional therapy for pulmonary infections includes intravenous (IV) or oral administration of antibiotics. However, in order to reach deep lung airways in sufficient concentrations, high doses of antibiotics are needed to be given systemically leading to either poor therapeutic action and/or potential toxic effects. Inhalation therapy can deliver drugs directly at the site of action and create an increased and more sustained local concentration leading to an increase in the therapeutic index and efficacy and a reduction of toxicities and the time of onset for the administered drug(s). Moreover, increased local concentrations can lead to improved diffusion of drug within the local lung environment. Among the various approaches for pulmonary delivery, dry powder inhalers (DPIs) provide an advantageous platform due to their ease of use and lack of propellants. Several drugs have already been approved by the U.S. Food and Drug Administration (FDA) for inhalation therapy.

Tigecycline is a broad-spectrum antibiotic that has shown activity against a wide variety of bacteria including Stenotrophomonas maltophilia (S. maltophilia), Streptococcus pneumonia, non-tuberculous mycobacteria and Staphylococcus aureus. There is a growing need for improved treatment of nosocomial and community acquired gram-negative bacteria in human patients. For example, S. maltophilia is a rod-shaped ubiquitous bacterium that is a leading multidrug resistant pathogen in hospitals around the world, known mostly to infect patients in intensive care units (ICU) thereby contributing to hospital acquired pneumonia. S. maltophilia has been shown to be more than 90% susceptible to tigecycline. However, there are complications due to the drug's chemical instability, high dose requirements and the question of specific targeting of infected regions. Thus, a more effective formulation of tigecycline should be developed.

Therapeutically, tigecycline is currently only available as a lyophilized powder for reconstitution in solvent and injection for the treatment of complicated abdominal and skin infections as well as community acquired bacterial pneumonia. However, tigecycline in this formulation, marketed as Tygacil®, is stable for only 6-48 hours upon reconstitution.

### SUMMARY

Determining ways to expand stability of tigecycline would advance its therapeutic application to better treat bacterial infections. Reports of inhaled tigecycline either use unstable and inconvenient aqueous based systems or use a dry powder inhaler that requires large excipient particles to be present in the powder formulation. These excipients and formulations are undesirable because the materials used as excipients also function as nutrients to the very microbes being treated.

Described herein is an effective inhaled tigecycline formulation. Specifically, tigecycline was developed as a dry powder formulation to enable local delivery of tigecycline at the site of infection within the lungs. The dry powder formulation overcomes tigecycline's chemical instability, dose requirements, and tissue targeting issues. The dry powder formulation also provides stability and administration convenience. In particular, the use of inhaled therapy eliminates the reconstitution step altogether, and the drug can be delivered in its stable dry form to the site of action. Additionally, dry powder inhalers are easy to use, patient friendly devices that do not require complicated patient maneuver. Inhalers also have an added advantage of being non-invasive, making it ideal from the point of view of patient acceptability, in particular for pediatric patients.

Described herein is a composition comprising a micronized tigecycline having a Dv90 particle size of less than 7 μm, wherein the composition is a powder. The composition is carrier free, and free of any stabilizing excipients. In some embodiments, the micronized tigecycline has a Dv90 particle size of less than 6 μm. In some embodiments, the micronized tigecycline has a Dv90 particle size of less than 5 μm. In some embodiments, the micronized tigecycline has a Dv50 particle size of less than 3 μm. In some embodiments, the micronized tigecycline has a Dv50 particle size of less than 2 μm. In some embodiments, the mean particle size of the micronized tigecycline in the composition is from 0.5 μm to 5 μm.

In some embodiments, the composition is formulated for pulmonary administration. In some embodiments, the micronized tigecycline is deliverable by aerosolization, a dry powder inhaler, a metered dose inhaler, an insufflator, or a dry powder nasal spray.

In some embodiments, a fine particle fraction of the micronized tigecycline is at least or about 70%, such as 75%, 80%, 85%, 90%, 95%, or greater. In some embodiments, the micronized tigecycline exhibits a specific surface area of at least or about 23 m²/g, such as 23 m²/g, 24 m²/g, 25 m²/g, 26 m²/g, 27 m²/g, 28 m²/g, 29 m²/g, or 30 m²/g. In some embodiments, a fine particle dose of the micronized tigecycline is at least about 9.2 mg, such as 9.2 mg, 9.3 mg, 9.4 mg, 9.5 mg, 9.6 mg, 9.7 mg, 9.8 mg, 9.9 mg, 10 mg, or more. In some embodiments, the mass median aerodynamic diameter of the micronized tigecycline is less than or about 2.1 μm, such as 2.1 μm, 2.0 μm, 1.9 μm, 1.8 μm, 1.7 μm, 1.6 μm, 1.5 μm, 1.4 μm, 1.3 μm, 1.2 μm, 1.0 μm, or less.

In some embodiments, the composition is a storage stable composition. In some embodiments, the composition is stable at a temperature of at least or about 25° C. for at least or about one month. In some embodiments, the composition is stable at a temperature of at least or about 25° C. for at least or about six months. In some embodiments, the composition is stable at a temperature of at least or about 25° C. and a relative humidity (RH) of about 60% for at least or about one month. In some embodiments, the composition is stable at a temperature of at least or about 25° C. and a relative humidity (RH) of about 60% for at least or about six months. In some embodiments, the composition is stable at a temperature of at least or about 40° C. for at least or about one month. In some embodiments, the composition is stable at a temperature of at least or about 40° C. for at least or about six months. In some embodiments, the composition is stable at a temperature of at least or about 40° C. and a relative humidity (RH) of about 75% for at least or about one month. In some embodiments, the composition is stable at a temperature of at least or about 40° C. and a relative humidity (RH) of about 75% for at least or about six months.

Described herein is a method of treating a pulmonary infection in a subject, comprising administering to a subject a composition of micronized tigecycline. In some embodiments, the micronized tigecycline may be delivered using a dry powder inhaler, a pressurized metered dose inhaler, an insufflator, a powder nebulizer, or a nasal spray, or specifically a dry powder nasal spray. In some embodiments, the dry powder inhaler is a handihaler or a plastiape. In some embodiments, a first dose of the micronized tigecycline is from about 5 mg to about 15 mg, such as from 5 mg to 6 mg, from 6 mg to 7 mg, from 7 mg to 8 mg, from 8 mg to 9 mg, from 9 mg to 10 mg, from 10 mg to 11 mg, from 11 mg to 12 mg, from 12 mg to 13 mg, from 13 mg to 14 mg, or from 14 mg to 15 mg. In some embodiments, any maintenance dose of the micronized tigecycline is from about 1 mg to about 5 mg, such as from 5 mg to 6 mg, from 6 mg to 7 mg, from 7 mg to 8 mg, from 8 mg to 9 mg, from 9 mg to 10 mg, from 10 mg to 11 mg, from 11 mg to 12 mg, from 12 mg to 13 mg, from 13 mg to 14 mg, or from 14 mg to 15 mg.

In some embodiments, the pulmonary infection is a gram-negative bacterial infection. In some embodiments, the gram-negative bacterial infection is a Stenotrophomonas maltophilia infection. In some embodiments, the gram-negative bacterial infection is a Burkholderia cepacia infection. In some embodiments, the pulmonary infection is a gram-positive bacterial infection. In some embodiments, the gram-positive bacterial infection is a Streptococcus pneumonia infection. In some embodiments, the gram-positive bacterial infection is a Staphylococcus aureus infection. In some embodiments, the gram-positive bacterial infection is a non-tuberculosis Mycobacterium infection.

In some embodiments, the method of treating a pulmonary infection in a patient further comprises administering one or more additional active agents to the subject. In some embodiments, the one or more additional active agents comprises one or more of an anti-inflammatory agent, an anti-bacterial agent, an anti-fungal agent, an anti-viral agent, an antiparasitic agent, or a combination thereof. In some embodiments, the one or more additional active agents is an anti-bacterial agent. In some embodiments, the active agent is a micronized powder. In some embodiments, the one or more additional compositions are administered concomitantly or sequentially with the composition of micronized tigecycline. The composition can be administered by local delivery, pulmonary delivery, and/or inhalation.

### BRIEF DESCRIPTION OF THE DRAWINGS

FIG. 1 illustrates a truncated set-up of a lab-scale Air Jet Mill.

FIG. 2 shows two graphs for (left) differential scanning calorimetry (DSC) thermograms of tigecycline powders at baseline and day 30 and (right) powder X-ray diffraction (PXRD) diffractograms of tigecycline powders at baseline and day 30.

FIG. 3 shows a graph of percent inhibition of Mycobacterium abscessus in the absence of tigecycline (control) and in the presence of tigecycline at different concentrations.

FIG. 4 shows two graphs depicting dispersion profiles indicated by (left) Dv50 and (right) Dv90 for milled and unmilled tigecycline powders at varying pressures from 0.5-3 bar where data is represented as mean±SD (n=3).

FIG. 5A shows two scanning electron micrographs of milled and unmilled tigecycline powders at 1000× magnification.

FIG. 5B shows two scanning electron micrographs of milled and unmilled tigecycline powders at 3000× magnification.

FIGS. 6A, 6B, and 6C show graphs illustrating the stability characterization of tigecycline before and after processing as (FIG. 6A) differential scanning calorimetry (DSC) thermograms of milled and unmilled powders at baseline (FIG. 6B) powder X-ray diffraction (PXRD) diffractograms of milled and unmilled powders at baseline, and (FIG. 6C) fourier-transform infrared spectroscopy (FTIR) of milled and unmilled powders at baseline.

FIG. 7A is a thermogravimetric analysis (TGA) graph of milled and unmilled powders at baseline.

FIG. 7B shows two dynamic vapor sorption (DVS) graphs for unmilled powders at baseline (top) and milled powders at baseline (bottom).

FIG. 8 shows a graph of fine particle fraction (FPF) of tigecycline powders at baseline and day 30 (* indicates a statistically significant difference (p<0.05) from milled tigecycline at day 0).

FIG. 9 shows graphs illustrating Next Generation Impactor (NGI) measured aerosol performance for (A) milled and unmilled powder at 4 kPa, (B) milled and unmilled powder at 1 kPa, (C) milled and unmilled powder using a dry powder inhaler, and (D) milled powder using a high resistance device.

FIG. 10 shows two graphs illustrating the stability of tigecycline (A) before aerosolization and (B) after aerosolization.

FIG. 11 shows two graphs illustrating results from the 2,5-diphenyl-2H-tetrazolium bromide (MTT) assay of unmilled tigecycline powders at (left) 4 hours and (right) 24 hours.

FIG. 12 shows a graph depicting transepithelial/transendothelial electrical resistance (TEER) measurements of the air-liquid interface (ALI) cell culture of Calu-3 cells.

FIG. 13 is a graph showing apical to basolateral permeability of unmilled tigecycline.

FIG. 14A is a bar graph illustrating antimicrobial efficacy of tigecycline in a planktonic S. maltophilia culture.

FIG. 14B is a plot showing biofilm biomass quantification over days as a function of absorbance vs. days.

FIG. 14C is a bar graph of MBEC concentration quantification of tigecycline by XTT assay where the y-axis represents absorbance and the x-axis represents different concentrations of tigecycline.

### DETAILED DESCRIPTION

Described herein is a novel inhaled tigecycline formulation that overcomes the drug's chemical instability, dose requirements, and tissue targeting issues by engineering tigecycline into a dry powder for inhalation. The current dosage of tigecycline, in its reconstituted form, for abdominal and skin infections requires 100 mg of tigecycline as the initial dose, followed by 50 mg every 12 hours. In contrast, local delivery of the developed dry powder formulation described herein has reduced the dose to as low as about 10 mg for the initial dose followed by lower dosages thereafter. Also, the tigecycline inhalable dry powder is stable upon storage for at least or about six months. In its dry powder inhaler form, tigecycline has superior aerosol performance and drug stability.

Described herein is a composition comprising a micronized tigecycline having a Dv90 particle size of less than or about 7 μm. The composition is a powder, is carrier free, and is free of any stabilizing excipients. As used herein, “micronized” refers to a reduced average diameter tigecycline using an air jet mill to reach a respirable particle size range. The respirable particle size range is defined as a particle size between about 1 and about 5 μm. As used herein a “carrier free” composition means that the composition can include less than or about 0.5%, less than or about 0.1%, less than or about 0.01%, less than or about 0.001%, less than or about 0.0001%, or 0% of the carrier. In some examples, a carrier is or includes lactose (e.g., inhalation grade lactose), ammonium alginate, calcium carbonate, calcium lactate, calcium phosphate, dibasic anhydrous, dibasic dehydrate, tribasic, calcium silicate, calcium sulfate, cellulose powdered, silicified microcrystalline, cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, anhydrous, monohydrate and corn starch, monohydrate and microcrystalline cellulose, spray dried, magnesium carbonate, magnesium oxide, maltodextrin, maltose, and/or other suitable inert materials used as inhalation carriers. In some cases, a carrier may refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. As used herein a “stabilizing excipient” refers to additives for formulations to slow down or prevent compound aggregation driven by stabilizing forces, destabilization of the denatured state, and which generally aids in extending the longevity of a compound.

The formulations described herein include micronized tigecycline having a mean particle size in a sufficient range to ensure it is effectively delivered to the target area of the lungs. As used herein, “mean particle size” refers to the volume median diameter as determined by laser diffraction when the powder particles were dispersed using at least or about 0.5 bar air pressure. In some embodiments, the mean particle size of the micronized tigecycline in the composition, based on measurements taken using a RODOS powder disperser, is from about 0.5 μm to about 5 μm. The mean particle size can be measured in terms of the Dv90 value or the Dv50 value, for example. As used herein, the parameter Dv90 corresponds to the cut-off size (preferably in μm) of the particles in a volume weighted distribution, which represents 90% of the total volume of the sample, and which have a particle size equal to or smaller than the Dv90 value. As used herein, Dv50 corresponds to the cut-off size (preferably in μm) of the particles in a volume weighted distribution, which represents 50% of the total volume of the sample, and which have a particle size equal to or smaller than the Dv50 value. In some embodiments, the micronized tigecycline has a Dv90 mean particle size of less than or about 6 μm. In some embodiments, the micronized tigecycline has a Dv90 mean particle size of less than or about 5 μm. In some embodiments, the micronized tigecycline has a Dv50 mean particle size of less than or about 3 μm. In some embodiments, the micronized tigecycline has a Dv50 mean particle size of less than or about 2 μm.

In some embodiments, the composition is formulated for pulmonary administration. As used herein, pulmonary administration refers to local or regional delivery of a drug to a subject to treat one or multiple diseases of the lungs and the respiratory tract. In some embodiments, the composition is formulated for inhalation. In some embodiments, the composition is formulated for intranasal administration. In some embodiments, the composition is formulated for mucosal administration. In some embodiments, the micronized tigecycline is deliverable by aerosolization, a dry powder inhaler, a metered dose inhaler, an insufflator, or a dry powder nasal spray.

The formulations described herein include micronized tigecycline having a fine particle fraction in a percent range to demonstrate effective delivery to the target area of the lungs. As used herein, “fine particle fraction” refers to the amount of drug that is made available to the deep lungs for therapeutic action which is irrespective of flow rate. In other words, the “fine particle fraction” or “FPF” can correspond to a deep lung dose. As used herein, “irrespective of flow rate” describes a fine particle fraction that does not change even in situations where, for example, the subject suffers from a lung disorder that causes the subject to have a low inspiratory flow capacity. In some embodiments, a fine particle fraction of the micronized tigecycline is at least or about 50%. In some embodiments, a fine particle fraction of the micronized tigecycline is at least or about 55%. In some embodiments, a fine particle fraction of the micronized tigecycline is at least or about 60%. In some embodiments, a fine particle fraction of the micronized tigecycline is at least or about 65%. In some embodiments, a fine particle fraction of the micronized tigecycline is at least or about 70%. In some embodiments, a fine particle fraction of the micronized tigecycline is at least or about 75%. In some embodiments, a fine particle fraction of the micronized tigecycline is at least or about 80%. In some embodiments, a fine particle fraction of the micronized tigecycline is at least or about 84%.

The formulations described herein include micronized tigecycline having a specific surface area (SSA) in a range sufficient for effective delivery and action in the target area of the lungs. As used herein, “specific surface area” refers to the total surface of a material per unit mass. In some embodiments, the micronized tigecycline exhibits a SSA of at least or about 13 m²/g. In some embodiments, the micronized tigecycline exhibits a SSA of at least or about 14 m²/g. In some embodiments, the micronized tigecycline exhibits a SSA of at least or about 15 m²/g. In some embodiments, the micronized tigecycline exhibits a SSA of at least or about 16 m²/g. In some embodiments, the micronized tigecycline exhibits a SSA of at least or about 17 m²/g. In some embodiments, the micronized tigecycline exhibits a SSA of at least or about 18 m²/g. In some embodiments, the micronized tigecycline exhibits a SSA of at least or about 19 m²/g. In some embodiments, the micronized tigecycline exhibits a SSA of at least or about 20 m²/g. In some embodiments, the micronized tigecycline exhibits a SSA of at least or about 21 m²/g. In some embodiments, the micronized tigecycline exhibits a SSA of at least or about 22 m²/g. In some embodiments, the micronized tigecycline exhibits a SSA of at least or about 23 m²/g.

The formulations described herein include micronized tigecycline having a fine particle dose in a sufficient range to ensure it is effectively delivered to the target area of the lungs. As used herein, “fine particle dose” refers to the dose (in mg) of an active ingredient (e.g., tigecycline) below a specified minimum aerodynamic size. In some embodiments, a fine particle dose of the micronized tigecycline is at least or about 7.4 mg. In some embodiments, a fine particle dose of the micronized tigecycline is at least or about 8.0 mg. In some embodiments, a fine particle dose of the micronized tigecycline is at least or about 8.5 mg. In some embodiments, a fine particle dose of the micronized tigecycline is at least 9.0 mg. In some embodiments, a fine particle dose of the micronized tigecycline is at least or about 9.2 mg.

The formulations described herein include micronized tigecycline having a mass median aerodynamic diameter (MMAD) in a sufficient range to ensure it is effectively delivered to the target area of the lungs. As used herein, MMAD describes the particle size based on the aerodynamic properties of the respective particles, in particular its settling behavior. The MMAD is preferably determined by regression analysis from the results of the Next Generation Impactor (NGI) studies. The MMAD relates to the median aerodynamic diameter of the particle distribution and is the diameter of a unit density sphere having the same settling velocity, in air, as the particle. The MMAD, thus, typically describes the particle size, preferably in micrometers (μm), with 50% of the particles in the distribution having a larger aerodynamic size and 50% of the particles in the distribution having a smaller aerodynamic size than the MMAD value. In some embodiments, the MMAD of the micronized tigecycline is less than or about 2.1 μm. In some embodiments, the MMAD of the micronized tigecycline is less than or about 5 μm. In some embodiments, the MMAD of the micronized tigecycline is less than or about 4 μm. In some embodiments, the MMAD of the micronized tigecycline is less than or about 3 μm. In some embodiments, the MMAD of the micronized tigecycline is less than or about 2.5 μm.

In some embodiments, the composition is a storage stable composition. In some embodiments, the composition is stable at a temperature of at least or about 25° C. for at least or about one month. In some embodiments, the composition is stable at a temperature of at least or about 25° C. for at least or about six months. In some embodiments, the composition is stable at a temperature of at least or about 25° C. and a relative humidity (RH) of about 60% for at least or about one month. In some embodiments, the composition is stable at a temperature of at least or about 25° C. and a relative humidity (RH) of about 60% for at least or about six months. In some embodiments, the composition is stable at a temperature of at least or about 40° C. for at least or about one month. In some embodiments, the composition is stable at a temperature of at least or about 40° C. for at least or about six months. In some embodiments, the composition is stable at a temperature of at least or about 40° C. and a relative humidity (RH) of about 75% for at least or about one month. In some embodiments, the composition is stable at a temperature of at least or about 40° C. and a relative humidity (RH) of about 75% for at least or about six months. In some embodiments, tigecycline is a light sensitive colored compound. In some embodiments, tigecycline is packaged in aluminum blisters. In some embodiments, tigecycline is packaged in amber vials. In some embodiments, tigecycline is packaged in vials, bottles or blisters that protect against light and moisture.

Described herein is a method of treating a pulmonary infection in a subject, comprising administering to a subject a composition of micronized tigecycline. The composition can be administered by local delivery, pulmonary delivery, and/or inhalation. In some embodiments, the micronized tigecycline may be delivered using a dry powder inhaler, a pressurized metered dose inhaler, an insufflator, or a dry powder nasal spray. In some embodiments, the dry powder inhaler is a handihaler or a plastiape. In some embodiments, a first dose of the micronized tigecycline is from about 5 mg to about 15 mg. In some embodiments, the first dose of the micronized tigecycline is 5 mg, 6 mg, 7 mg, 8 mg, 9 mg, 10 mg, 11 mg, 12 mg, 13 mg, 14 mg, or 15 mg. In some embodiments, any maintenance dose of the micronized tigecycline is from about 1 mg to about 5 mg. As used herein, a “maintenance dose” is the dose administered throughout a dosage regimen to maintain effective drug concentrations after the first dose. In some embodiments, a maintenance dose of the micronized tigecycline can be 1 mg, 2 mg, 3 mg, 4 mg, or 5 mg.

Described herein is a method for treating a pulmonary infection in a subject. As used herein, a “pulmonary infection” is an infectious lung disease caused by the action of an infectious agent (e.g., a virus, bacteria, or fungi). In some embodiments, the pulmonary infection is a gram-negative bacterial infection. In some embodiments, the gram-negative bacterial infection is a Stenotrophomonas maltophilia infection. In some embodiments, the gram-negative bacterial infection is a Burkholderia cepacia infection. In some embodiments, the gram-negative bacterial infection is an Acinetobacter infection (e.g., an Acinetobacter baumannii infection), a Pseudomonas infection (e.g., a Pseudomonas aeruginosa infection), a Klebsiella infection, an Escherichia infection, a Salmonella infection, a Yersinia infection, a Shigella infection, a Proteus infection, an Enterobacter infection, a Serratia infection, or a Citrobacter infection.

In some embodiments, the pulmonary infection is a gram-positive bacterial infection. In some embodiments, the gram-positive bacterial infection is a Streptococcus pneumonia infection. In some embodiments, the gram-positive bacterial infection is a Staphylococcus aureus infection. In some embodiments, the gram-positive bacterial infection is a non-tuberculosis Mycobacterium infection. In some embodiments, the gram-positive bacterial infection is a Bacillus infection, a Listeria infection, a Streptococcus infection (e.g., a Streptococcus pyogenes infection), an Enterococcus infection, or a Clostridium infection. In some embodiments, the infection is caused by anaerobes.

In some embodiments the infection is caused by one or more of the following organisms: Staphylococcus aureus, methicillin susceptible Staphylococcus aureus, methicillin resistant Staphylococcus epidermidis, methicillin susceptible Staphylococcus epidermidis, methicillin resistant Streptococcus pneumoniae, penicillin sensitive Streptococcus pneumoniae, penicillin intermediate Streptococcus pneumoniae, penicillin resistant Streptococcus pyogenes, Enterococcus faecalis, Enterococcus faecium, Acinetobacter spp., Burkholderia cepacian, Citrobacter freundii, Enterobacter aerogenes, Enterobacter cloacae, Escherichia coli, Klebsiella pneumoniae, Moraxella catarrhalis, Morganella morganii, Proteus mirabilis, Pseudomonas aeruginosa, Stenotrophomonas maltophilia, Serratia marcescens, Haemophilus influenzae, Bacteroides fragilis, Clostridium difficile, Clostridium perfringens, Peptostreptococcus spp., Chlamydia pneumoniae, Legionella pneumoniae, and Mycoplasma pneumoniae.

In some embodiments, the method of treating a pulmonary infection is a patient further comprises administering one or more additional active agents to the subject. In some embodiments, the one or more additional active agents may exhibit synergistic action with tigecycline. In some embodiments, the one or more additional active agents comprises one or more of an anti-inflammatory agent, an anti-bacterial agent, an anti-fungal agent, an anti-viral agent, an antiparasitic agent, or a combination thereof. In some embodiments, the one or more additional active agents may treat two or more bacteria. In some embodiments, the one or more additional active agents is an anti-bacterial agent. In some embodiments, the active agent is a micronized powder. In some embodiments, the one or more additional compositions are administered concomitantly or sequentially with the composition of micronized tigecycline.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Micronization of Tigecycline (TIG)

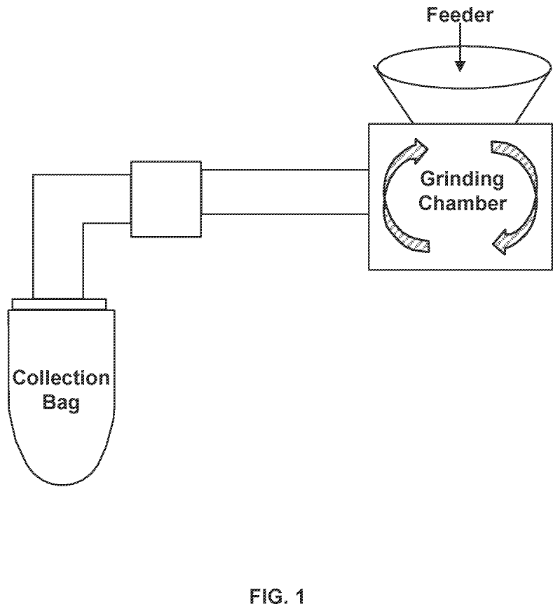

Tigecycline was micronized using a lab-scale Air Jet Mill (Jet-O-Mizer, Fluid Energy, Telford, PA). The air jet mill was used in a truncated version to reduce the losses as shown in FIG. 1. The air jet mill was set at 75 psi grind pressure, 65 psi feed pressure, and 1 g/min feed rate. Approximately, 5 g of powder was milled during each run to attain a particle size distribution in the respirable range between 0.5-5 μm. The micronized powder was collected from the collection bag and stored in amber vials for further analysis at 4° C. Micronized tigecycline powder was recovered from the collection bag of the Air Jet Mill with a yield of 85%.

### Example 2: Particle Size

HELOS laser diffraction instrument (Sympatec Americas, Pennington, NJ) using the RODOS dispersion feature was used to assess the geometric particle size distribution of both the milled and unmilled powders. The particle size distribution was measured at different pressures from 0.5-3 bar to analyze the effect of dispersion pressure on particle deaggregation. Measurements that were between 5% and 25% optical density were averaged to determine particle size distribution.

### Example 3: Morphology

The morphology of milled and unmilled tigecycline was analyzed by Scanning Electron Microscopy (SEM). The powders were mounted on SEM stubs and sputter-coated with 12 nm of platinum/palladium (Pt/Pd). Imaging was performed using a FEI Quanta 650 ESEM (FEI Company, Hillsboro, OR).

### Example 4: Measuring the Impact of Processing on Tigecycline Powders

### Differential Scanning Calorimetry (DSC)

Thermograms of both milled and unmilled tigecycline were obtained by heating the samples at a ramp rate of 10° C./min from 25-350° C. using an Auto Q20 (TA Instruments-Waters LLC, New Castle, DE, USA) Differential Scanning calorimeter (DSC). The nitrogen flow rate was maintained at 50 mL/min. Approximately 4 mg of each sample was loaded in standard DSC pans (DSC Consumables Inc., Austin, MN, USA), which were crimped using a Tzero sample press (TA Instruments-Waters LLC, New Castle, DE, USA).

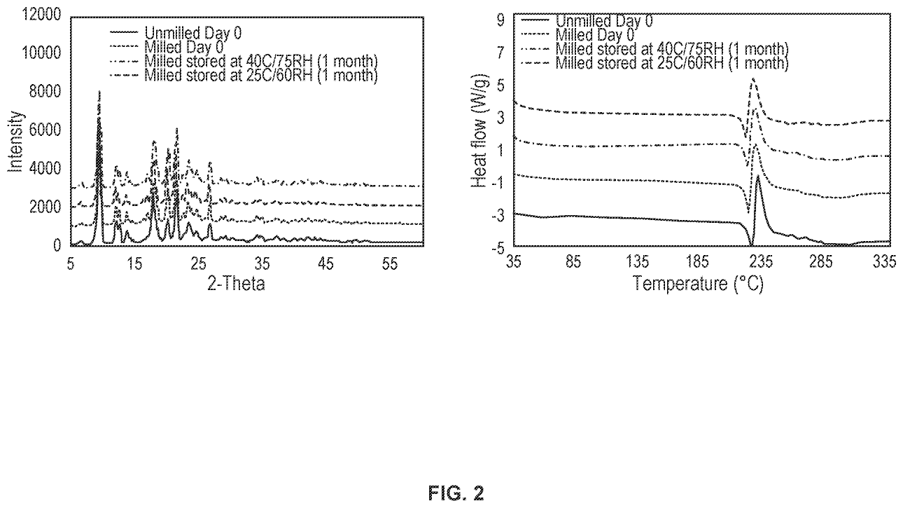

DSC thermograms of the milled powders, in comparison to the unmilled powders, remained the same, both after processing and upon storage of 25° C./60% RH or 40° C./75% RH for one month. Thus, tigecycline remained crystalline upon processing and prolonged storage at intermediate and accelerated conditions (FIG. 2, left).

### X-Ray Powder Diffraction

To analyze the crystallinity of milled powders as compared to unmilled powders, X-ray powder diffraction (XRPD) was used. Rigaku MiniFlex 600 II (Rigaku Corporation, Tokyo, Japan) was used to obtain diffractograms of unmilled and milled tigecycline powders. The target radiation of copper was set at 40 kV voltage and 40 mA current. The samples were scanned from 5-60 degrees at a ramp rate of 2 degrees/minute and a step size of 0.02.

PXRD diffractograms of the milled powders, in comparison to the unmilled powders, remained the same, both after processing and upon storage of 25° C./60% RH or 40° C./75% RH for one month. Thus, tigecycline remained crystalline upon processing and prolonged storage at intermediate and accelerated conditions (FIG. 2, right).

### Example 5: Fourier Transform Infrared Radiation (FTIR)

FTIR was used to assess peak shifts that may occur as a result of degradation using iS50 FT-IR with a SMART OMNI-Sampler, Nicolet, (ThermoFisher Scientific, Waltham, MA, USA). Briefly, 20-25 mg of sample was analyzed for absorbance from 700-4000 cm⁻¹ wavenumber. The resolution was 4 cm⁻¹ and the background was run before each sample.

### Example 6: Water Content

The water content of the powders was assessed using thermogravimetric analysis (TGA). About 5 mg of powder was loaded into a 70 μL alumina crucible. The samples were heated from 35-350° C. at a rate of 10° C./min. The point at which the weight change in the sample showed a plateau before the commencement of degradation was used to calculate the percent water content.

### Example 7: Water Sorption

Dynamic vapor sorption (DVS) analysis of the powders was performed using an Automated Water Sorption Analyzer (Surface Measurement Systems, London, UK). Briefly, 5-10 mg of milled and unmilled TIG powder was used as the sample. The samples were allowed to equilibrate under nitrogen until dm/dt<0.002%. Water sorption was analyzed as 10 steps from 0% relative humidity (RH) to 100% RH where step dm/dt was set at 0.075% at 10% increments in RH.

### Example 8: Specific Surface Area (SSA)

The Monosorb Gas Adsorption unit (Quantachrome, Boynton Beach, FL, USA) was used to measure the specific surface area (SSA) of the powders. A single point Braummer-Emmett-Teller (BET) method was utilized. Milled or unmilled tigecycline powder was outgassed under helium at 40° C. for 24 h before gas adsorption analysis. The choice of this outgassing temperature was to reduce the risk of heat-related degradation of the powders, while still promoting water vapor removal. Adsorption-desorption cycles were repeated until consecutive surface area measurements differed by less than 5% such that complete removal of impurities was ensured. The resulting surface area value was then normalized to the sample weight to obtain the SSA.

### Example 9: True Density

The true density of the processed powders was measured using a helium multipycnometer (Quantachrome Instruments, Boynton Beach, FL) with a micro-sample cell. The sample cell was filled to at least 75% of the total volume with milled or unmilled tigecycline powder. Using equation (1), the pressure of both the reference cell (P1) and sample cell (P2) was determined, and the true volume of the sample was calculated as follows:

$$V p = V c - V R [ ( P 1 / P 2 ) - 1 ] \qquad ( 1 )$$

For the micro-sample cell utilized, Vc was calibrated at 11.76156 cm³ and VR=6.34216 cm³. The sample weight was divided by the measured true volume to obtain the true density. Measurements were repeated until the standard deviation of ten consecutive true density measurements was less than 0.01 g/mL.

### Example 10: In-Vitro Aerosol Performance

The in vitro aerosol performance of the milled and unmilled powders was assessed using Next Generation Impactor (NGI) with a low and high resistance DPI (RS01 Monodose DPI (RS01 DPI; Plastiape). About 10 mg of the powder was added to size 3 hydroxypropyl methylcellulose (HPMC) capsules and the analysis was performed for a length of time sufficient to draw 4 L of air through the NGI, as per USP requirements. The pressure drop across the system was equivalent to either 4 kPa or 1 kPa. The flow rates required across the pressure drop to draw 4 L of air through the NGI, as per USP requirements, were 93 L/min for 4 kPa and 47.6 L/min for 1 kPa. The aerosol performance was compared between the low resistance device and the high resistance device. Also, the performance of the Plastiape devices was compared to that of Handihaler, a commercially used DPI. TIG deposited in each stage was dissolved in a buffer containing 0.015 M oxalic acid and 0.015 M sodium phosphate monobasic at pH 7 and quantified using a UV-Visible spectrophotometer (Tecan Systems, Inc., San Jose, CA, USA) at 245 nm. The aerodynamic particle size metrics that were calculated include emitted dose (ED), emitted fraction (EF), the respiratory fraction (RF), fine particle dose (FPD), fine particle fraction (FPF), mass median aerodynamic diameter (MMAD), and geometric standard deviation (GSD).

### Example 11: Stability Upon Aerosolization

Stability upon aerosolization was assessed using High-Performance Liquid Chromatography (HPLC) by the method described by Lucelia Magalhaes da Silva and Herida Regina Nunes Salgado in Validation of a Stability-Indicating RP-LC Method for the Determination of Tigecycline in Lyophilized Powder, J. Chromatogr. Sci. 51:192-199 (2012). The powder accumulated on Stage 4 (after aerosolization), as well as the capsule (before aerosolization) after running the NGI, was dissolved in a buffer containing 0.015 M oxalic acid and 0.015 M sodium phosphate monobasic at pH 7. The mobile phase used was 75 (0.015 M oxalic acid and 0.015 M sodium phosphate monobasic at pH 7): 25 (Acetonitrile). The flow rate was kept at 1 mL/min and UV absorbance was measured at 245 nm. A forcefully degraded sample (tigecycline left in water for 1 week at RT without protection from light) was also incorporated as a control to assess the ability of the method to detect degradants.

### Example 12: Storage Stability

The milled powder was stored for 30 days at 25° C./60% RH and 45° C./75% RH, as specified by the International Council for Harmonization (ICH) intermediate and accelerated stability conditions using sealed amber vials. At the 30-day and 6 month time point, the powder was characterized for its physicochemical properties, stability, and aerosol performance. The results obtained from day 30 and 6 months were compared to baseline measurements to declare storage stability.

### Example 13: Cell Culture

Calu-3 cells between passage 14-17 were grown in Dulbecco's Modified Eagle Medium (DMEM)+10% Fetal Bovine Serum (FBS)+1% Non-Essential Amino Acids (NEAA)+1% Penicillin Streptomycin (PS)+1% Sodium pyruvate in a 75 cm² flask. The media was changed every other day.

### Cell Viability

To assess the toxicity of the tigecycline powders, Calu-3 cells were seeded into 96-well tissue culture plates at a seeding density of 9000 cells/well. The cells were cultured overnight in media. The following day, tigecycline powders (unmilled) were added to the cells at concentrations ranging from 1 μM to 1 mM. The powders were dissolved in media and 100 μL of each concentration was added to the wells. Control wells containing only media were also set up. The cells were allowed to incubate with the treatment for 72 hours. At 72 hours after the addition of the dissolved powders, the media were removed, and the cells were incubated with an admixture of 50 μl of MTT (3-(4,5-Dimethylthiazol-2-yl)) reagent and 50 μl of the FBS-deprived culture media for 3 hours at 37° C. At 3 hours, MTT reagent/culture media was removed and 150 μl of MTT dissolving solvent was added to the well. The well-plate was wrapped in foil and kept on an electronic shaker for 15 minutes. The absorbance was then read at 590 nm using the Tecan microplate reader (Tecan Global Headquarters, Männedorf, Switzerland).

### Example 14: Air Liquid Interface (ALI) Culture

Calu-3 cells were plated at a cell density of 33,000 cells/well in 24-well transwell inserts on the apical side and 500 μL media was added to the basolateral side. 48 hours after plating the cells, the cells were airlifted (i.e the media from the apical and basolateral side was aspirated and only the basolateral side was replaced with media).

### Cell Integrity

Cell integrity of the Calu-3 monolayer was measured every other day after airlifting. Briefly, the media from the basolateral side was aspirated. 200 μL of Hank's balanced salt solution (HBSS) buffer was added to the apical side and 500 μL of HBSS was added to the basolateral side. The transepithelial electrical resistance (TEER) was measured using an epithelial Voltohmmeter. The short electrode was dipped into the buffer on the apical side and the longer electrode was dipped into the buffer on the basolateral side. Using the measured value the TEER was calculated using equation (2):

$$TEER = measured electrical resistance * surface area ( 0.33 ) \qquad ( 2 )$$

### Drug Permeability

The permeation experiments were carried out between days 5-7 after airlifting when the TEER values were ˜700Ω which indicates epithelial cell integrity and the absence of a leaky monolayer. The amount of drug transported across the cell layer were measured as follows: Milled and unmilled powders of tigecycline were dissolved in 200 μL of HBSS at a concentration of 10 μM and 20 μM. The media in the basolateral side was replaced by HBSS. 10 μL sample was collected from the basolateral side at the following intervals: 5 min, 15 min, 30 min, 45 min, 60 min, 2 hours, 4 hours, 6 hours, 8 hours, 12 hours, and 24 hours. After each aliquot, 10 μL of HBSS buffer was replaced into the basolateral chamber. This procedure was repeated to measure the permeability from the basolateral side to the apical side where samples were aliquoted from the apical side. The apparent permeability can be calculated as follows:

$$Papp = \frac{dQ * 1 * 1}{Dt A {C}_{0}} \qquad ( 3 )$$

Where
dQ/dt=Concentration flux
A=Surface area
Cₒ=initial concentration

### Example 15: S. maltophilia

### Bacterial Culture

Stenotrophomonas maltophilia was obtained from American Type Culture Collection (ATCC, Manassas, VA) (ATCC 13637). S. maltophilia dried pellet was reconstituted in nutrient broth and frozen until further use. An inoculation loop was used to collect S. maltophilia from the stock solution and was streaked onto nutrient agar plates. The plates were incubated at 37° C. for 24 hours. At the 24-hour point, an inoculation loop was used to transfer bacteria from the plate to 5 mL of liquid nutrient broth. The bacteria in the liquid medium were left overnight on a shaker at 37° C.

In Vitro Assessment of Minimum Inhibitory Concentration (MIC) of TIG Powders Against S. maltophilia Bacteria

Liquid bacterial culture was prepared as described above. 200 μL of the overnight liquid culture was added to 96 well plates. Varying concentrations of TIG were added to the 96 well plates ranging from 0.12 μg/mL to 64 μg/mL. All TIG concentrations were tested in triplicate. The well plate was allowed to incubate at 37° C. for 24 hours, and the MIC was determined using the OD 600 values.

S. maltophilia Biofilm Growth Conditions

S. maltophilia was grown as described above. 200 μL of the bacteria in nutrient broth was added onto 96 well plates for varying amounts of time ranging from 1 day to 5 days. The bacterial biomass at each time point was assessed using crystal violet assay. Briefly, the liquid media was aspirated at each time point, and the wells were washed twice with 200 μL of phosphate-buffered saline (PBS). To the cleaned wells, 100 μL of 0.05% w/v of crystal violet in water was added and allowed to sit for 10 minutes. At 10 minutes, the crystal violet solution was aspirated, and the wells were washed twice with PBS to remove the excess dye. The bound dye was then solubilized in 33% v/v acetic acid, and the absorbance was measured at 595 nm. All measurements were conducted in triplicate.

In Vitro Assessment of Minimum Biofilm Eradication Concentration (MBEC) of TIG Powders Against S. maltophilia Bacteria

200 μL of this standardized culture was added to 96 well plates and allowed to grow overnight at 37° C. After, the media was aspirated and replaced with 100 μL media containing varying concentrations of TIG from 0.1 to 2.5 μM. S. maltophilia was incubated with TIG for 24 hours. At 24 hours, the viability of the bacteria was evaluated using the XTT assay. Briefly, 25 μL of XTT solution was added to each of the wells and allowed to shake on a benchtop shaker for 3 hours. After 3 hours, the absorbance was measured at 490 nm.

### Example 16: In Vitro Assessment of Efficacy of TIG Against Mycobacterium abscessus

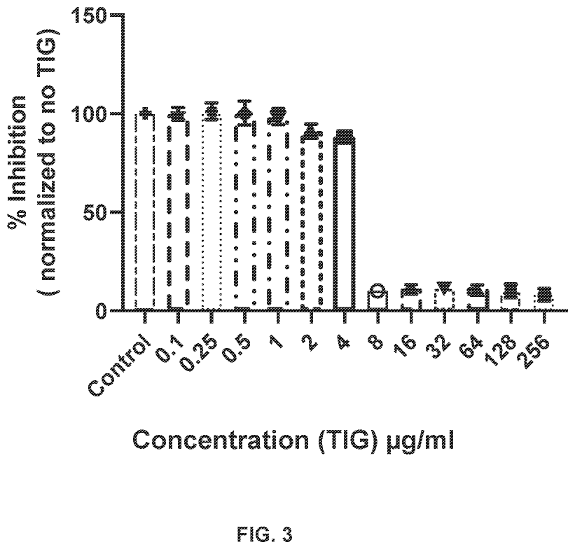

Mab was cultured in Middlebrook 7H9 liquid broth for 48 hours. 100 μL of inoculum was then added to 96 well plate with 50 μL of different concentrations of Tigecycline (0.1 to 240 μg/mL) (FIG. 3). The minimal inhibitory concentration (MIC) of Tigecycline against Mab was found to be greater than about 4 μg/mL, such as about 8 μg/mL.

### Example 17: Impact of Processing on Particle Size and Morphology

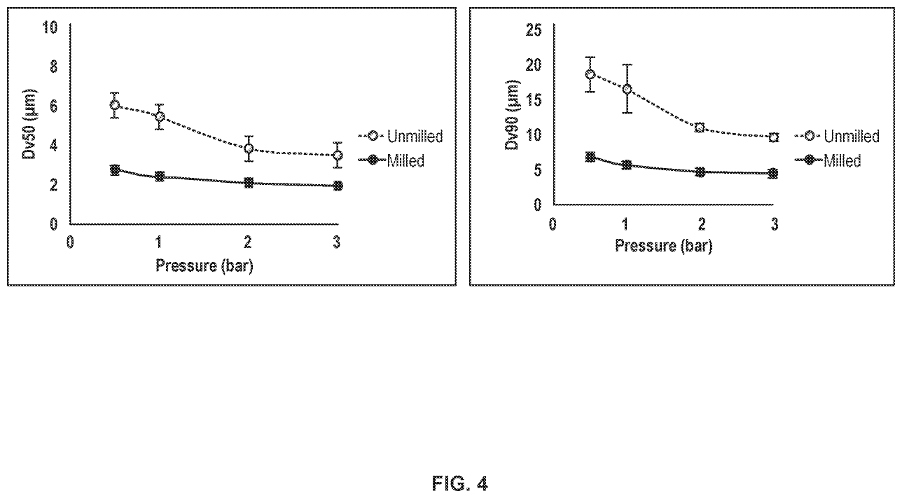

The dispersion profiles of milled and unmilled tigecycline powders were analyzed using laser diffraction. For unmilled tigecycline, as the dispersing pressure increased the Dv50 and Dv90 decreased while at lower dispersing pressures of 0.5 bar the powders showed a relatively larger particle size distribution (FIG. 4). This indicates the pressure-dependent deaggregation of the unmilled powders. In contrast, the milled powders showed little to no differences in Dv50 and Dv90 across the pressure range indicating pressure independent deaggregation. The particle size distribution for the milled powders remains the same across the pressure range (FIG. 4). The particle size (Dv90) of the milled powder was below 7 μm irrespective of the particle sizing dispersing pressure while the unmilled powder exhibited a pressure dependent decrease in particle size (at higher dispersion pressures, the particle size was lower). However, even at a dispersing pressure of 3 bar, the unmilled powders had a particle size (Dv90) of 9.48 μm (Table 1).

TABLE 1
90^{th} percentile tigecycline powder particle size at different pressures
Dv90 (μm)

| | |
|---|---|
| Milled | Unmilled |

| | | | | |
|---|---|---|---|---|
| Pressure | Avg. | SD | Avg. | SD |

| | | | | | |
|---|---|---|---|---|---|
| 0.5 | bar | 6.67 | 0.26 | 18.38 | 2.41 |
| 1 | bar | 5.5 | 0.72 | 16.29 | 3.45 |
| 2 | bar | 4.55 | 0.38 | 10.79 | 0.44 |
| 3 | bar | 4.37 | 0.42 | 9.49 | 0.52 |

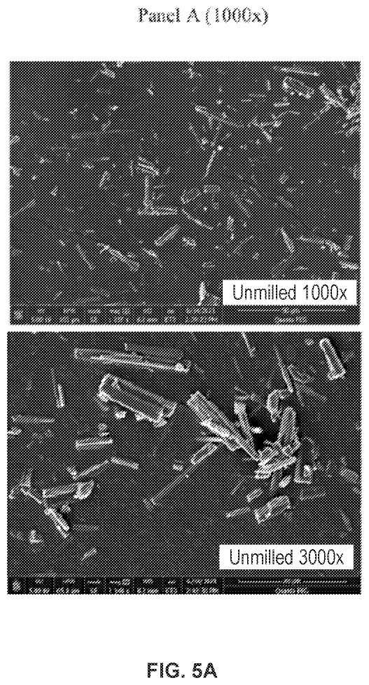

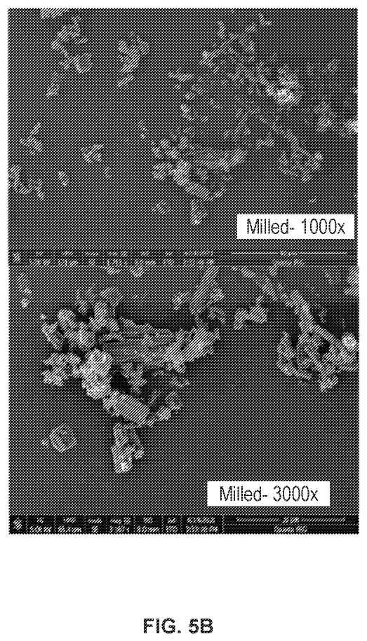

SEM images of unmilled powders revealed the needle-shaped morphology of tigecycline as seen in FIG. 5A. Milled powders retained the needle-shaped morphology but were broken into much smaller particles as compared to unmilled powder (FIG. 5B). The fracture of the particles during jet milling led to a reduction in the length of these particles, however, the edges and width of the particles remain similar to unmilled powders.

### Example 18: Impact of Processing on Stability

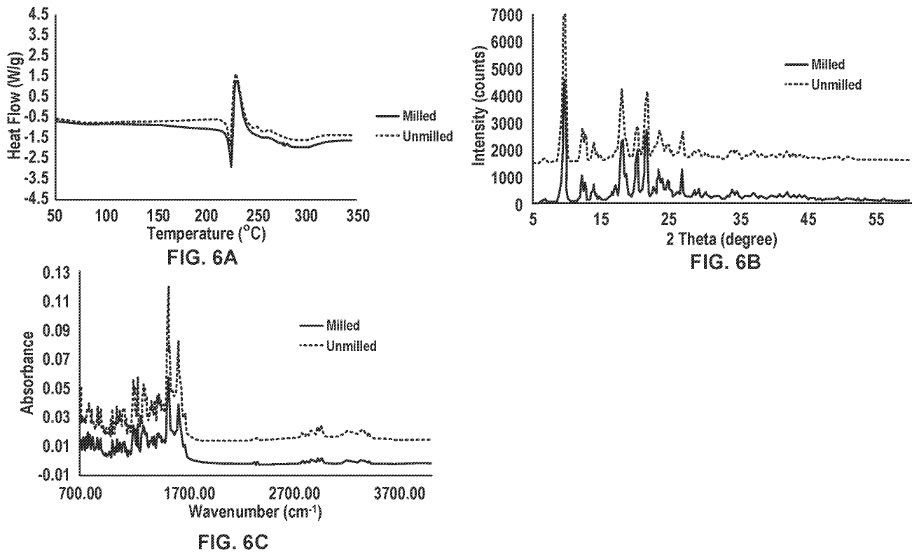

Differential scanning calorimetry (DSC) thermogram of unmilled powder revealed a sharp endothermic peak at 225° C. which is characteristic of the melting point of tigecycline (Form VI). Tigecycline is characterized by degradation upon melting which was observed in the case of unmilled powders. The milled powder retained the thermal profile of unmilled powder indicating the absence of any form change or formation of degradation products (FIG. 6A). The X-ray powder diffraction (XRPD) profile of unmilled powder revealed that the Tigecycline powder was crystalline after processing. The diffractograms of milled powders were identical to that of the unmilled powder indicating the absence of form change as a result of the micronization process (FIG. 6B).

Fourier transform infrared spectroscopy (FTIR) analyses of milled and unmilled tigecycline were performed to identify any peak shifts that may occur as a result of degradation by oxidation of tigecycline. However, the FTIR scans revealed no differences in the profiles of the milled and unmilled powders indicating that the oxidative stability of the powder was preserved during the micronization process (FIG. 6C).

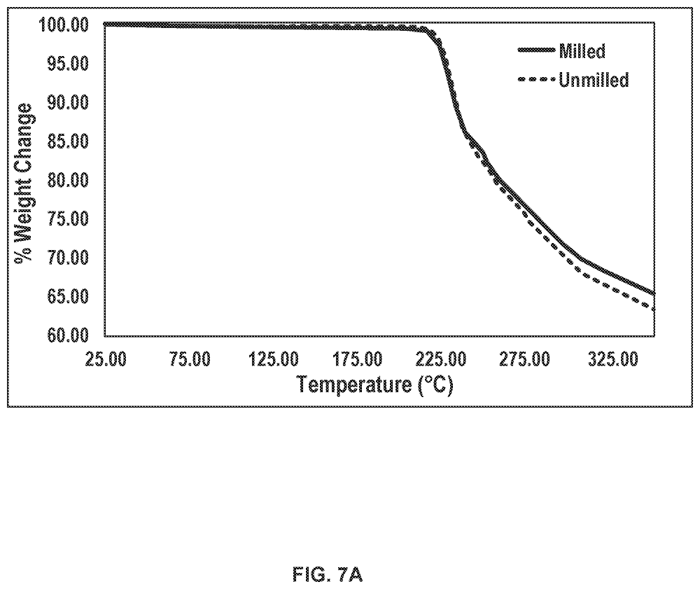

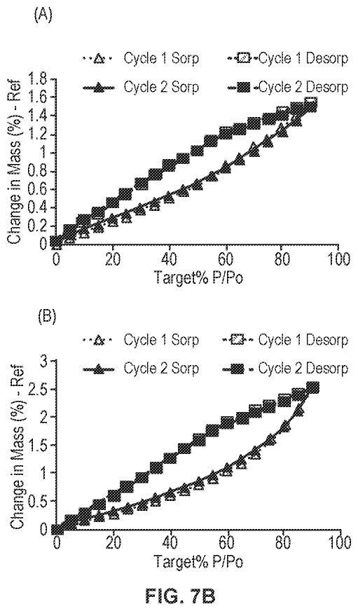

Thermogravimetric analysis (TGA) plots of the milled and unmilled powders revealed no thermal events of note. Water content was negligible in both milled and unmilled powders. The data obtained from the TGA agreed with the DSC thermograms and was characterized by degradation upon melting after 230° C. (FIG. 7A). DVS revealed that both milled and unmilled tigecycline powders were not hygroscopic. The powders exhibited less than 2.5% of water absorption after a full sorption-desorption cycle as see in FIG. 7B. This result was in agreement with the data obtained from TGA.

### Example 19: Impact of Processing on Powder Properties

Specific Surface Area (SSA) data of the milled powders was twice as much of the unmilled powders. These correlate well with the scanning electron microscope (SEM) images which revealed that micronization of tigecycline conferred particles that had a fractured needle-like morphology. Since the originally long, needle-like particles are each broken into several smaller particles the surface area of the milled powders was bound to increase with micronization. True Density (TD) of the milled and unmilled tigecycline powders, on the other hand were comparable with no statistically significant differences observed (Table 2).

TABLE 2
Tigecycline Powder Properties

| Parameter | Unmilled | Milled |
|---|---|---|
| True Density (g/mL) | 1.282 ± 0.0004 | 1.289 ± 0.15 |
| Specific Surface Area (m²) | 12.76 ± 0.15 | 23.8 ± 0.04* |

*Indicates a significant difference when compared to unmilled TIG (p < 0.05)

### Example 20: Aerosol Performance of TIG Powders

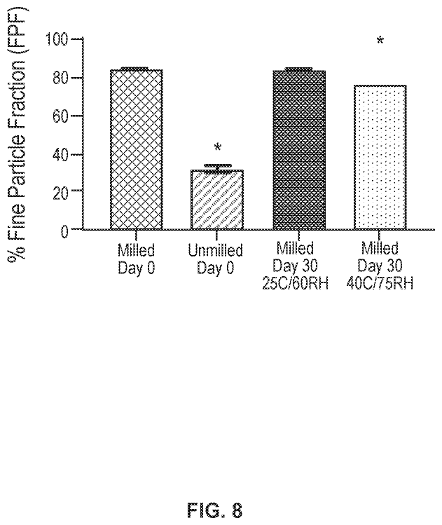

Unmilled powder of tigecycline exhibited poor aerosol performance with >65% of the emitted dose being deposited in the capsule, device, throat and early NGI stages. The unmilled powders exhibited a cohesive nature and failed to fully disperse from the capsule despite a pressure drop of 4 kPa. The milled powders, on the other hand, exhibited excellent aerosol properties wherein the fine particle fraction (FPF) of the powders were 84.77±0.03% which was significantly higher than that of the unmilled powders that had an FPF of 31.70±1.83%. Upon storage for one month at intermediate conditions (25° C./60% RH), the FPF remained comparable at 83.93±0.71%. However, upon storage at accelerated conditions (40° C./75% RH) the FPF reduced by almost 8% to 76.69±0.02% (FIG. 8). The FPF is defined as a percentage of particles below 5 μm with respect to the emitted dose.

The significant differences in FPF also translated to enormous differences in the Fine Particle Dose (FPD), where unmilled powders had an FPD of only 2.57±0.06 mg while milled powders exhibited an FPD about four-times higher, at 8.96±0.03 mg. Of note, upon storage for one month at accelerated conditions (40° C./75% RH) the FPD remained at 8.36±0.01 mg which is still within the estimated lung dose required for antibacterial efficacy. This is in contrast with the results for FPF under accelerated conditions. The mass-median aerodynamic diameter (MMAD) of unmilled tigecycline was 5.73±0.52 μm which was three-fold higher than that of milled tigecycline with a MMAD of 1.90=0.16 μm.

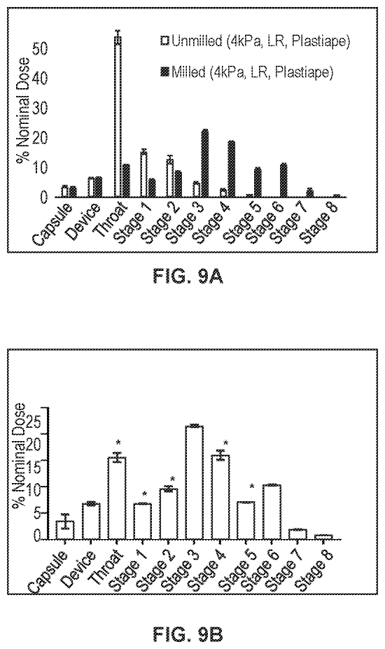

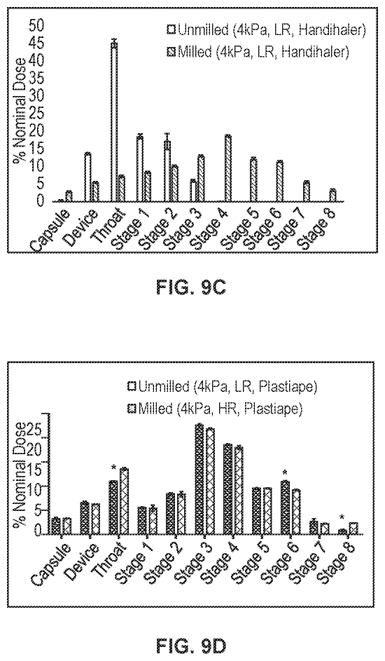

It should be noted that milled tigecycline maintained its aerosol properties even at a lower pressure drop of 1 kPa, wherein the FPF and FPD were found to be 84.78=0.03% and 8.98±0.02 mg respectively. Milled powders also retained their performance when dispersed from different device types as well as when exposed to different resistance devices. When dispersed from a high resistance Plastiape device, tigecycline had a FPF of 85.02±0.03% that translated into a FPD of 8.09±0.01 mg. Additionally, when the aerosol performance was tested in a commercially available Handihaler device, the FPF and FPD were slightly lower as compared to that of the Plastiape. However, the FPD was 7.41±0.006, which was not significantly different from that of Plastiape. The aerosol performance metrics including emitted dose (ED), emitted fraction (EF), mass median aerodynamic diameter (MMAD), geometric standard deviation (GSD) and respirable fraction (RF), defined as the percent of particles below 5 μm with respect to the total dose, have been summarized in Table 3 while the percent deposition of TIG under different conditions and devices has been summarized in FIG. 9.

TABLE 3
Aerodynamic Particle Size Distribution (APSD) metrics.

| | Emitted | Emitted | MMAD | | RF |
|---|---|---|---|---|---|
| | Dose (mg) | Fraction (%) | (μm) | GSD | (<5 μm) |
| Unmilled | 8.48 ± 0.25 | 90.20 ± 0.27 | 5.73 ± 0.52 | 1.25 ± 0.01 | 28.59 ± 1.56 |
| 4 kPa | | | | | |
| LR Plastiape | | | | | |
| Milled | 10.30 ± 0.02 | 90.15 ± 0.12 | 1.90 ± 0.16 | 1.29 ± 0.001 | 69.96 ± 0.17 |
| 4 kPa | | | | | |
| LR Plastiape | | | | | |
| Milled | 10.22 ± 0.03 | 92.28 ± 0.23 | 1.01 ± 0.11 | 1.74 ± 0.06 | 72.14 ± 0.08 |
| 1 kPa | | | | | |
| LR Plastiape | | | | | |
| Milled | 9.23 ± 0.02 | 91.34 ± 0.06 | 1.81 ± 0.11 | 1.39 ± 0.002 | 77.66 ± 0.08 |
| 4 kPa | | | | | |
| HR | | | | | |
| Plastiape | | | | | |
| Unmilled 4 | 7.66 ± 5.11 | 64.60 ± 0.23 | 8.27 ± 1.41 | 1.13 ± 0.11 | 16.44 ± 2.12 |
| kPa | | | | | |
| Handihaler | | | | | |
| Milled | 8.10 ± 0.23 | 66.60 ± 1.87 | 1.73 ± 0.06 | 1.54 ± 0.02 | 54.54 ± 0.06 |
| 4 kPa | | | | | |
| Handihaler | | | | | |

### Example 21: Stability Upon Aerosolization

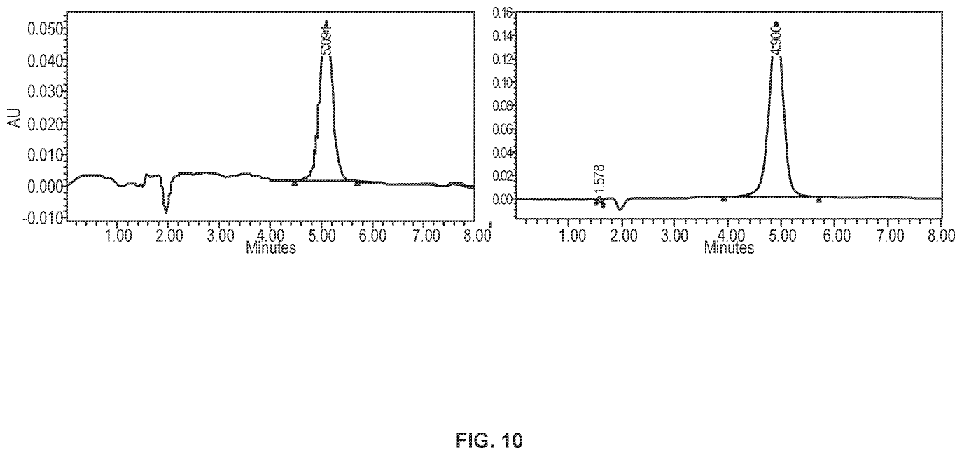

To assess the feasibility of administering tigecycline via a DPI inhaler, it is important to confer stability upon aerosolization. Comparison of the HPLC chromatograms of samples collected from the capsule and stage 4 revealed no additional peaks in the stage 4 sample. The method was validated to be able to detect degradation by hydrolysis, oxidation, epimerization and photodegradation. However, none of the degradation peaks were observed in either of the samples indicating that the stability of tigecycline was completely preserved despite aerosolization (FIG. 10).

### Example 22: Impact of Storage on Stability

Tigecycline powders stored in intermediate (25° C./60% RH) and accelerated (40° C./75% RH) conditions when analyzed using DSC exhibited the same thermal events as that seen in milled and unmilled powders at baseline. PXRD analysis of the stored samples revealed that the powders remained crystalline, and FTIR analysis revealed no peak shifts indicating that the powders remained stable upon storage. Finally, HPLC analysis of the powders confirmed that the powders are stable upon storage indicated by the absence of any additional peaks. The storage stability of tigecycline powders is summarized in Table 4.

TABLE 4
Storage stability of milled tigecycline at intermediate
and accelerated conditions in comparison to baseline.

| | | Milled TIG | Milled TIG | Milled TIG | Milled TIG |
|---|---|---|---|---|---|
| | | (Stored at | (Stored at | (Stored at | (Stored at |
| | Milled TIG | 25 C/60% RH | 40 C/75% RH | 25 C/60% RH | 40 C/75% RH |
| | (Baseline) | for 1 month) | for 1 month) | for 6 month) | for 6 month) |
| FPF | 84.78 ± 0.03 | 83.93 ± 0.71 | 76.70 ± 0.02 | 79.86 ± 0.88 | 73.53 ± 0.15 |
| (%) | | | | | |
| DSC | 225 | 225 | 225 | 225 | 225 |
| (Tg ° C.) | | | | | |
| TGA | No water | No water | No water | No water | No water |
| | content | content | content | content | content |
| XRD | Crystalline | Crystalline | Crystalline | Crystalline | Crystalline |
| HPLC | No peak | No peak | No peak | No peak | No peak |
| | shift/No | shift/No | shift/No | shift/No | shift/No |
| | reduction in | reduction in | reduction in | reduction in | reduction in |
| | concentration | concentration | concentration | concentration | concentration |
| FTIR | No peak shift | No peak shift | No peak shift | No peak shift | No peak shift |
| DVS | <3% moisture | <3% moisture | <3% moisture | <3% moisture | <3% moisture |
| | sorption | sorption | sorption | sorption | sorption |

### Example 23: Impact of Tigecycline on Cell Viability, Integrity, and Permeability

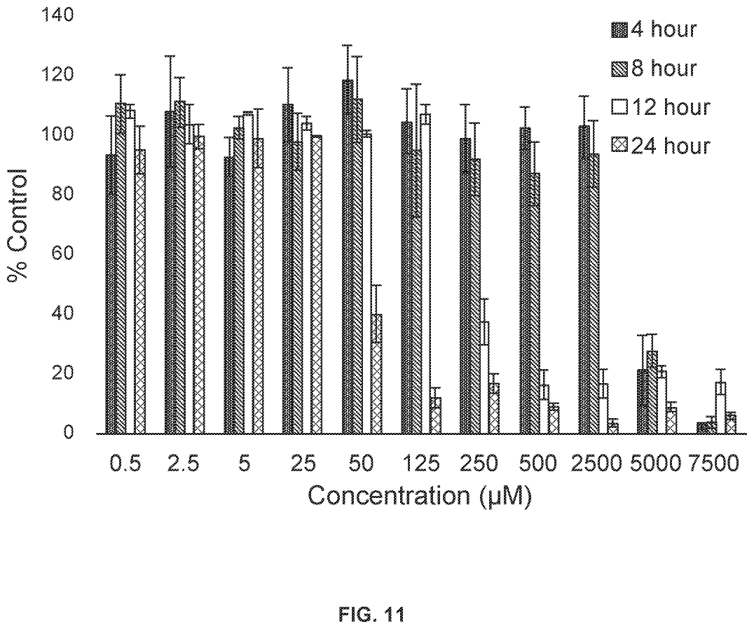

Calu-3 cells exhibited a concentration and time dependent cell viability profile in submerged culture. At the 4-hour and 8-hour point, Calu-3 cells were viable up to 2.5 mM concentration of tigecycline. However, at 5 mM and 7.5 mM, significant cell death was observed. At the 12-hour point, the maximum concentration of tigecycline that allowed for the cells to maintain their viability was 125 μM while at 24 hours it was 25 μM (FIG. 11). Based on these results, 100 μM of tigecycline was used to assess the permeability across the Calu-3 monolayer.

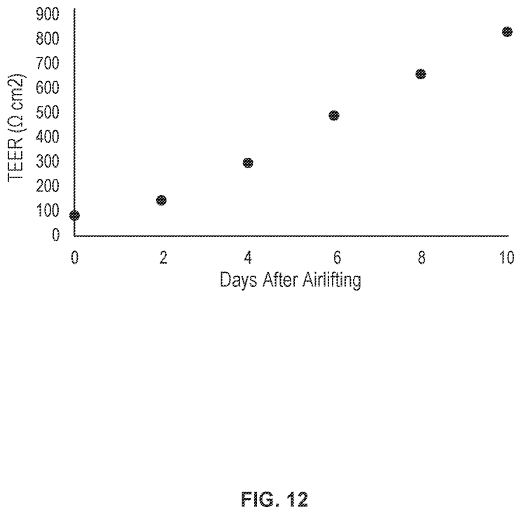

The TEER measurement of the Calu-3 monolayer during the differentiation period of the ALI culture revealed a linear increase in the TEER values up to day 8 (FIG. 12). The permeability experiments were conducted when the TEER values were ˜700Ω (between days 5-7). The TEER values measured before the start of the permeability assessment and after the completion of the experiment were found to be comparable, indicating that the Calu-3 cell monolayer remained intact throughout the experiment.

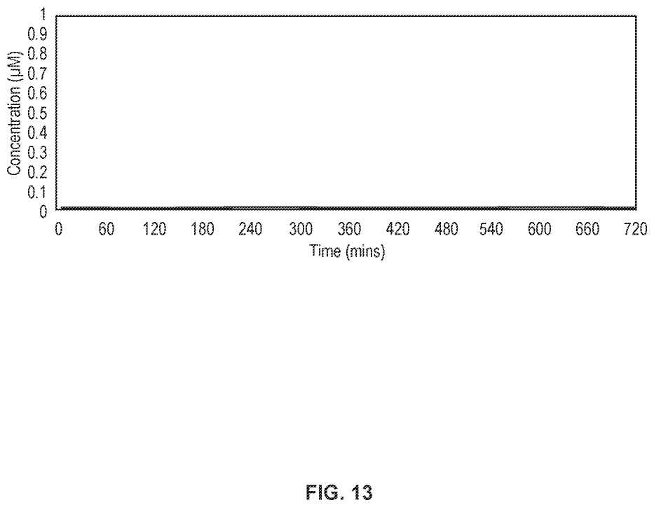

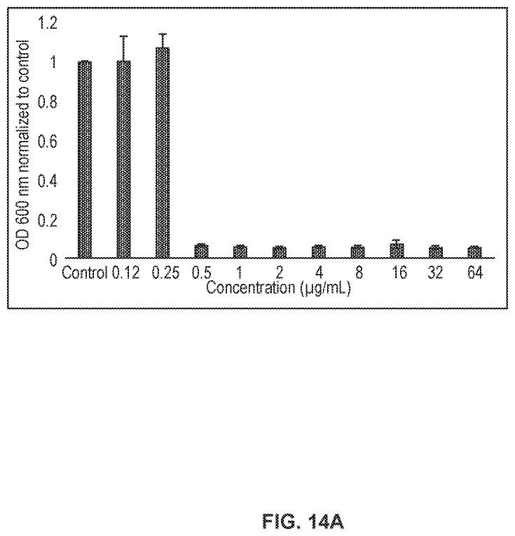

Finally, the permeability of tigecycline across the Calu-3 cell monolayer measured from the apical to basolateral revealed that less than 1 μM of tigecycline permeated through the cell monolayer across a 12-hour time point. At each time point, no peak for tigecycline was detected in a HPLC method with the limit of detection 1 μM. Analysis of the amount of tigecycline in donor side at the end of the experiment revealed that nearly ˜100% of the drug still remained in the apical side. This trend of impermeability of tigecycline across the cell monolayer was maintained even in the basolateral to apical direction (FIG. 13).

### Example 24: Efficacy of Milled Tigecycline Powders Against S. maltophilia in an In Vitro System

### Minimum Inhibitory Concentration

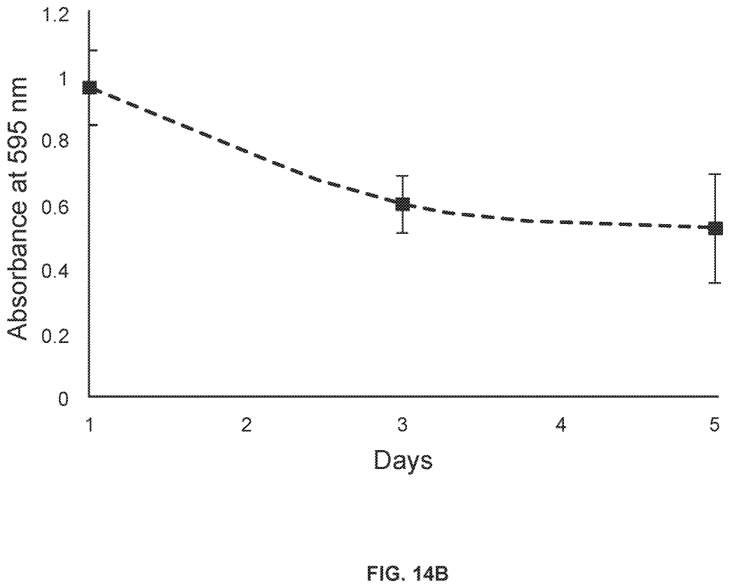

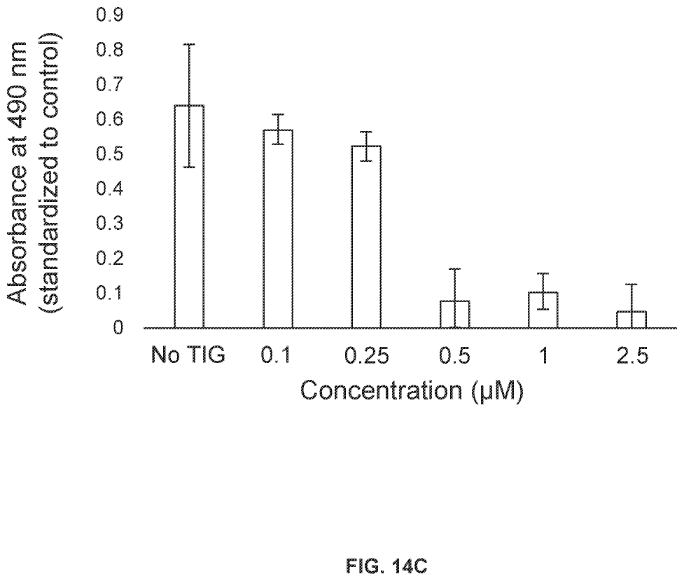

Tigecycline powder inhibited growth of S. maltophilia, grown in TSP, completely at concentrations equal to or greater than 0.5 μg/mL. At 0.1 μg/mL and 0.25 μg/mL, tigecycline failed to inhibit bacterial growth. Thus, the Minimum Inhibitory Concentrations (MIC) of tigecycline was found to be 0.5 μg/mL. FIG. 14A depicts the OD 600 values for all groups normalized to control (where no drug was added). At concentrations 0.12 and 0.25 μg/mL that OD 600 values were similar to that of control and hence the normalized value was ˜1. At concentrations >0.5 μg/mL, the OD 600 values were significantly lower than that of control.

### Biofilm Growth and Minimum Biofilm Eradication Concentration

The biofilm biomass quantification using crystal violet assay was conducted on days 1, 3, and 5. The absorbance values at 595 nm revealed that the maximum biofilm growth was observed 24 hours after inoculation of the culture in the 96 well plates. Thus, the bacteria undergo log phase growth within 24 hours. At days 3 and 5, the bacterial biofilm biomass is slightly lower and continues to be consistent on both days (FIG. 14B). Based on these results, the biofilm eradication efficiency of TIG was assessed at 24 hours.

XTT assay revealed that at concentrations above 0.5 μM, TIG could inhibit biofilm formation by nearly 85%. However, at concentrations lower than 0.5 μM, TIG could not effectively inhibit S. maltophilia biofilm formation. OD 600 values of S. maltophilia cultures exposed to varying concentrations of TIG also agreed with the XTT results (FIG. 14C).

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions are intended to fall within the scope of the appended claims. Thus, a combination of steps, elements, components, or constituents can be explicitly mentioned herein; however, all other combinations of steps, elements, components, and constituents are included, even though not explicitly stated.

### REFERENCES

PCT Patent Application Publication. Nos. 2008/155405; 2005/025536; 2006/099258; and 2008/0014256.
U.S. Pat. Nos. 9,107,929; 9,511,145; 9,694,078; and 9,855,335.
Pearce C, Ruth M M, Pennings L J, Wertheim H F, Walz A, Hoefsloot W, Ruesen C, Muñoz Gutiérrez J, Gonzalez-Juarrero M, van Ingen J. Inhaled tigecycline is effective against Mycobacterium abscessus in vitro and in vivo. Journal of Antimicrobial Chemotherapy. 2020 July; 75 (7): 1889-94.
Pearce C M, Walz A, Gonzalez-Juarrero M, Ali Z, Munoz-Gutierrez J, van Ingen J. Inhaled tigecycline induces change in immune cell modulation that synergizes with antimicrobial activity in chronic pulmonary Mycobacterium abscessus disease.
Pearce C. Inhalational antibiotic therapy for treatment of chronic pulmonary Mycobacterium abscessus disease in mice (Doctoral dissertation, Colorado State University).
Noskin G A. Tigecycline: A New Glycylcycline for Treatment of Serious Infections. Vol 41.; 2005.
Stein G E, Craig W A. Tigecycline: A Critical Analysis. Vol 43.; 2006.
Brunaugh A D, Jan S U, Ferrati S, Smyth H D C. Excipient-Free Pulmonary Delivery and Macrophage Targeting of Clofazimine via Air Jet Micronization. Molecular Pharmaceutics. 2017;14 (11): 4019-4031.
Yazdi A K, Smyth H D C. Carrier-free high-dose dry powder inhaler formulation of ibuprofen: Physicochemical characterization and in vitro aerodynamic performance. International Journal of Pharmaceutics. 2016; 511 (1): 403-414.
Kyu, H. H., et al., L. Age-Sex Differences in the Global Burden of Lower Respiratory Infections and Risk Factors, 1990-2019: Results from the Global Burden of Disease Study 2019. Lancet Infect Dis 2022, 22 (11), 1626-1647.
Schmidt, B. Z.; Haaf, J. B.; Leal, T.; Noel, S. Cystic Fibrosis Transmembrane Conductance Regulator Modulators in Cystic Fibrosis: Current Perspectives. Clin Pharmacol 2016, 8, 127-140.
Esposito, A.; Pompilio, A.; Bettua, C.; Crocetta, V.; Giacobazzi, E.; Fiscarelli, E.; Jousson, O.; Bonaventura, G. di. Evolution of Stenotrophomonas maltophilia in Cystic Fibrosis Lung over Chronic Infection: A Genomic and Phenotypic Population Study. Front Microbiol 2017, 8 (AUG), 1590.
McFarland, L. v.; Evans, C. T.; Goldstein, E. J. C. Strain-Specificity and Disease-Specificity of Probiotic Efficacy: A Systematic Review and Meta-Analysis. Front Med (Lausanne) 2018, 5 (MAY).
Steinkamp, G.; Wiedemann, B.; Rietschel, E.; Krahl, A.; Gielen, J.; Bärmeier, H.; Ratjen, F.; Staab, D.; Brömme, S.; Lindemann, H.; Heuer, H.; Posselt, H. G.; Dockter, G.; Schuster, A.; Nüßlein, T.; van Koeningsbruggen, S.; Bargon, J. Prospective Evaluation of Emerging Bacteria in Cystic Fibrosis. J Cyst Fibros 2005, 4 (1), 41-48.
Berg, G.; Martinez, J. L. Friends or Foes: Can We Make a Distinction between Beneficial and Harmful Strains of the Stenotrophomonas maltophilia Complex? Frontiers in Microbiology 2015, 6 (MAR), 241.
Gil-Gil, T.; Martinez, J. L.; Blanco, P. Mechanisms of Antimicrobial Resistance in Stenotrophomonas maltophilia: A Review of Current Knowledge. Expert Rev Anti Infect Ther 2020, 18 (4), 335-347.
Waters, V.; Atenafu, E. G.; Lu, A.; Yau, Y.; Tullis, E.; Ratjen, F. Chronic Stenotrophomonas maltophilia Infection and Mortality or Lung Transplantation in Cystic Fibrosis Patients. J Cyst Fibros 2013, 12 (5), 482-486.
Guerci, P.; Bellut, H.; Mokhtari, M.; Gaudefroy, J.; Mongardon, N.; Charpentier, C.; Louis, G.; Tashk, P.; Dubost, C.; Ledochowski, S.; Kimmoun, A.; Godet, T.; Pottecher, J.; Lalot, J. M.; Novy, E.; Hajage, D.; Bouglé, A.; Constantin, J. M.; Perbet, S. Outcomes of Stenotrophomonas maltophilia Hospital-Acquired Pneumonia in Intensive Care Unit: A Nationwide Retrospective Study. Crit Care 2019, 23 (1).
Said, M. S.; Tirthani, E.; Lesho, E. Stenotrophomonas maltophilia. StatPearls 2022.
Hand, E.; Davis, H.; Kim, T.; Duhon, B. Monotherapy with Minocycline or Trimethoprim/Sulfamethoxazole for Treatment of Stenotrophomonas maltophilia Infections. J Antimicrob Chemother 2016, 71 (4), 1071-1075.
Al-Jasser, A. M. Stenotrophomonas maltophilia Resistant to Trimethoprim-Sulfamethoxazole: An Increasing Problem. Ann Clin Microbiol Antimicrob 2006, 5 (1), 1-3.
Mojica, M. F.; Humphries, R.; Lipuma, J. J.; Mathers, A. J.; Rao, G. G.; Shelburne, S. A.; Fouts, D. E.; Duin, D. van; Bonomo, R. A. Clinical Challenges Treating Stenotrophomonas maltophilia Infections: An Update.
Brooke, J. S. Expert Review of Anti-Infective Therapy New Strategies against Stenotrophomonas maltophilia: A Serious Worldwide Intrinsically Drug-Resistant Opportunistic Pathogen.
Samonis, G.; Karageorgopoulos, D. E.; Maraki, S.; Levis, P.; Dimopoulou, D.; Spernovasilis, N. A.; Kofteridis, D. P.; Falagas, M. E. Stenotrophomonas maltophilia Infections in a General Hospital: Patient Characteristics, Antimicrobial Susceptibility, and Treatment Outcome. PLOS One 2012, 7.
Brooke, J. S. Stenotrophomonas maltophilia: An Emerging Global Opportunistic Pathogen. Clin Microbiol Rev 2012, 25 (1), 2-41. DOI: 10.1128/CMR.00019-11.
Wenzler, E.; Fraidenburg, D. R.; Scardina, T.; Danziger, L. H. Inhaled Antibiotics for Gram-Negative Respiratory Infections. Clin Microbiol Rev 2016, 29 (3), 581-632.
Borghardt, J. M.; Kloft, C.; Sharma, A. Inhaled Therapy in Respiratory Disease: The Complex Interplay of Pulmonary Kinetic Processes. Can Respir J 2018, 2018.
Nichols, D. P.; Durmowicz, A. G.; Field, A.; Flume, P. A.; Vandevanter, D. R.; Mayer-Hamblett, N. Identifying Patterns of Learner Behaviour: What Business Statistics Students Do with Learning Resources. 2017.
Agu, R. U.; Ugwoke, M. I.; Armand, M.; Kinget, R.; Verbeke, N. The Lung as a Route for Systemic Delivery of Therapeutic Proteins and Peptides. Respir Res 2001, 2 (4), 198.
Labiris, N. R.; Dolovich, M. B. Pulmonary Drug Delivery. Part II: The Role of Inhalant Delivery Devices and Drug Formulations in Therapeutic Effectiveness of Aerosolized Medications. Br J Clin Pharmacol 2003, 56 (6), 600.
Ye, Y.; Ma, Y.; Zhu, J. The Future of Dry Powder Inhaled Therapy: Promising or discouraging for Systemic Disorders? Int J Pharm 2022, 614, 121457.
Levy, M. L.; Carroll, W.; Izquierdo Alonso, J. L.; Keller, C.; Lavorini, F.; Lehtimäki, L. Understanding Dry Powder Inhalers: Key Technical and Patient Preference Attributes. Adv Ther 2019, 36 (10), 2547.
Mehta, P. Dry Powder Inhalers: A Focus on Advancements in Novel Drug Delivery Systems. J Drug Deliv 2016, 2016, 1-17.
Stein, G. E.; Craig, W. A. Tigecycline: A Critical Analysis. Clinical Infectious Diseases 2006, 43 (4), 518-524.
Maloney, S. E.; Alshiraihi, I. M.; Singh, A.; Stewart, I. E.; Mariner Gonzalez, J.; Gonzalez-Juarrero, M.; Meibohm, B.; Hickey, A. J. Spray Dried Tigecycline Dry Powder Aerosols for the Treatment of Nontuberculous Mycobacterial Pulmonary Infections. Tuberculosis (Edinb) 2023, 139, 102306.
Wallace, R. J.; Dukart, G.; Brown-Elliott, B. A.; Griffith, D. E.; Scerpella, E. G.; Marshall, B. Clinical Experience in 52 Patients with Tigecycline-Containing Regimens for Salvage Treatment of Mycobacterium Abscessus and Mycobacterium Chelonae Infections. Journal of Antimicrobial Chemotherapy 2014, 69 (7), 1945.
Goncalves-Vidigal, P.; Grosse-Onnebrink, J.; Mellies, U.; Buer, J.; Rath, P. M.; Steinmann, J. Stenotrophomonas maltophilia in Cystic Fibrosis: Improved Detection by the Use of Selective Agar and Evaluation of Antimicrobial Resistance. Journal of Cystic Fibrosis 2011, 10 (6), 422-427.
Yazdi, A. K.; Smyth, H. D. C. Carrier-Free High-Dose Dry Powder Inhaler Formulation of Ibuprofen: Physicochemical Characterization and in Vitro Aerodynamic Performance. Int J Pharm 2016, 511 (1), 403-414.
da Silva, L. M.; Salgado, H. R. N. Validation of a Stability-Indicating RP-LC Method for the Determination of Tigecycline in Lyophilized Powder. J Chromatogr Sci 2013, 51 (2), 192-199.
Fiegel, J.; Ehrhardt, C.; Schaefer, U. F.; Lehr, C.-M.; Hanes, J. Large Porous Particle Impingement on Lung Epithelial Cell Monolayers-Toward Improved Particle Characterization in the Lung; 2003.
Zhang, Y.; Mackenzie, B.; Koleng, J. J.; Maier, E.; Warnken, Z. N.; Williams, R. O. Development of an Excipient-Free Peptide Dry Powder Inhalation for the Treatment of Pulmonary Fibrosis. Mol Pharm 2020, 17 (2), 632-644.
Brodka-Pfeiffer, K.; Langguth, P.; Graß, P.; Häusler, H. Influence of Mechanical Activation on the Physical Stability of Salbutamol Sulphate. European Journal of Pharmaceutics and Biopharmaceutics 2003, 56 (3), 393-400.
Chamayou, A.; Dodds, J. A. Chapter 8 Air Jet Milling. Handbook of Powder Technology 2007, 12, 421-435.
Descamps, M.; Willart, J. F. Perspectives on the Amorphisation/Milling Relationship in Pharmaceutical Materials. Adv Drug Deliv Rev 2016, 100, 51-66.
Reutzel-Edens, S. M.; Newman, A. W. Physical Characterization of Hygroscopicity in Pharmaceutical Solids. Polymorphism: in the Pharmaceutical Industry 2006, 235-258.
Newman, A. W.; Reutzel-Edens, S. M.; Zografi, G. Characterization of the “Hygroscopic” Properties of Active Pharmaceutical Ingredients. J Pharm Sci 2008, 97 (3), 1047-1059.
Bagwan, N. U. S.; Sheokand, S.; Kaur, A.; Dubey, G.; Puri, V.; Bharatam, P. v.; Bansal, A. K. Role of Surface Molecular Environment and Amorphous Content in Moisture Sorption Behavior of Milled Terbutaline Sulphate. European Journal of Pharmaceutical Sciences 2021, 161, 105782.
Prudic, A.; Ji, Y.; Luebbert, C.; Sadowski, G. Influence of Humidity on the Phase Behavior of API/Polymer Formulations. Eur J Pharm Biopharm 2015, 94, 352-362.
Norderud Lærum, B.; Telg, G.; Stratelis, G. Need of Education for Dry Powder Inhaler Storage and Retention—a Patient-Reported Survey. Multidiscip Respir Med 2016, 11 (1).
Vandana, K. R.; Prasanna Raju, Y.; Harini Chowdary, V.; Sushma, M.; Vijay Kumar, N. An Overview on in Situ Micronization Technique—An Emerging Novel Concept in Advanced Drug Delivery. Saudi Pharmaceutical Journal: SPJ 2014, 22 (4), 283.
Shetty, N.; Cipolla, D.; Park, H.; Zhou, Q. T. Physical Stability of Dry Powder Inhaler Formulations. Expert Opin Drug Deliv 2020, 17 (1), 77.
Groß, R.; Berkenfeld, K.; Schulte, C.; Ebert, A.; Sule, S.; Sule, A.; Lamprecht, A. State of the Art in Capsule-Based Dry Powder Inhalers: Deagglomeration Techniques and the Consequences for Formulation Aerosolization. Pharmaceutics 2022, 14 (6).
Buttini, F.; Quarta, E.; Allegrini, C.; Lavorini, F. Understanding the Importance of Capsules in Dry Powder Inhalers. Pharmaceutics 2021, 13 (11).
Donovan, M. J.; Kim, S. H.; Raman, V.; Smyth, H. D. Dry Powder Inhaler Device Influence on Carrier Particle Performance. J Pharm Sci 2012, 101 (3), 1097-1107.
Molimard, M.; Kottakis, I.; Jauernig, J.; Lederhilger, S.; Nikolaev, I. Performance Characteristics of Breezhaler® and Aerolizer® in the Real-World Setting. Clin Drug Investig 2021, 41 (5), 415-424.
Clark, A. R.; Weers, J. G.; Dhand, R. The Confusing World of Dry Powder Inhalers: It Is All About Inspiratory Pressures, Not Inspiratory Flow Rates. J Aerosol Med Pulm Drug Deliv 2020, 33 (1), 1.
Dal Negro, R. W. Dry Powder Inhalers and the Right Things to Remember: A Concept Review. Multidiscip Respir Med 2015, 10 (1), 1-4.
Geller, D. E.; Madge, S. Technological and Behavioral Strategies to Reduce Treatment Burden and Improve Adherence to Inhaled Antibiotics in Cystic Fibrosis. Respir Med 2011, 105, S24-S31.
Briesacher, B. A.; Quittner, A. L.; Saiman, L.; Sacco, P.; Fouayzi, H.; Quittell, L. M. Adherence with Tobramycin Inhaled Solution and Health Care Utilization. BMC Pulm Med 2011, 11.
Greenwood, J.; Schwarz, C.; Sommerwerck, U.; Nash, E. F.; Tamm, M.; Cao, W.; Mastoridis, P.; Debonnett, L.; Hamed, K. Ease of Use of Tobramycin Inhalation Powder Compared with Nebulized Tobramycin and Colistimethate Sodium: A Crossover Study in Cystic Fibrosis Patients with Pulmonary Pseudomonas Aeruginosa Infection. Ther Adv Respir Dis 2017, 11 (7), 249-260.
Konstan, M. W.; Flume, P. A.; Kappler, M.; Chiron, R.; Higgins, M.; Brockhaus, F.; Zhang, J.; Angyalosi, G.; He, E.; Geller, D. E. Safety, Efficacy and Convenience of Tobramycin Inhalation Powder in Cystic Fibrosis Patients: The EAGER Trial. J Cyst Fibros 2011, 10 (1), 54-61.
Harrison, M. J.; McCarthy, M.; Fleming, C.; Hickey, C.; Shortt, C.; Eustace, J. A.; Murphy, D. M.; Plant, B. J. Inhaled versus Nebulised Tobramycin: A Real World Comparison in Adult Cystic Fibrosis (CF). J Cyst Fibros 2014, 13 (6), 692-698.
Anderson, S.; Atkins, P.; Cipolla, D.; Clark, A.; Daviskas, E.; Disse, B.; Entcheva-Dimitrov, P.; Fuller, R.; Gonda, I.; Lundb€, H.; Olsson, B.; Weers, J. Inhaled Medicines: Past, Present, and Future. 2022.
Sahakijpijarn, S.; Smyth, H. D. C.; Miller, D. P.; Weers, J. G. Post-Inhalation Cough with Therapeutic Aerosols: Formulation Considerations. Adv Drug Deliv Rev 2020, 165-166, 127-141.
Brunaugh, A. D.; Jan, S. U.; Ferrati, S.; Smyth, H. D. C. Excipient-Free Pulmonary Delivery and Macrophage Targeting of Clofazimine via Air Jet Micronization. Mol Pharm 2017, 14 (11), 4019-4031.
Magalhães Da Silva, L.; Salgado, H. R. N. Tigecycline. Ther Drug Monit 2010, 32 (3), 282-288.
Stigliani, M.; Haghi, M.; Russo, P.; Young, P. M.; Traini, D. Antibiotic Transport across Bronchial Epithelial Cells: Effects of Molecular Weight, LogP and Apparent Permeability. European Journal of Pharmaceutical Sciences 2016, 83, 45-51.
Labiris, N. R.; Dolovich, M. B. Pulmonary Drug Delivery. Part I: Physiological Factors Affecting Therapeutic Effectiveness of Aerosolized Medications. UK BJCPBritish Journal of Clinical Pharmacology 1365, 56, 6588599.
Hastedt, J. E.; Backman, P.; Cabal, A.; Clark, A.; Ehrhardt, C.; Forbes, B.; Hickey, A. J.; Hochhaus, G.; Jiang, W.; Kassinos, S.; Kuehl, P. J.; Prime, D.; Son, Y. J.; Teague, S.; Tehler, U.; Wylie, J. IBCS: 1. Principles and Framework of an Inhalation-Based Biopharmaceutics Classification System. Mol Pharm 2022.
Farrell, D. J.; Sader, H. S.; Jones, R. N. Antimicrobial Susceptibilities of a Worldwide Collection of Stenotrophomonas maltophilia Isolates Tested against Tigecycline and Agents Commonly Used for S. maltophilia Infections. Antimicrob Agents Chemother 2010, 54 (6), 2735-2737.
Rossi, F.; Andreazzi, D. Overview of Tigecycline and Its Role in the Era of Antibiotic Resistance. The Brazilian Journal of Infectious Diseases 2006, 10 (3), 203-216.
Pearce, C.; Ruth, M. M.; Pennings, L. J.; Wertheim, H. F. L.; Walz, A.; Hoefsloot, W.; Ruesen, C.; Muñoz Gutiérrez, J.; Gonzalez-Juarrero, M.; van Ingen, J. Inhaled Tigecycline Is Effective against Mycobacterium Abscessus in Vitro and in Vivo. J Antimicrob Chemother 2020, 75 (7), 1889-1894.
Rodvold, K. A.; George, J. M.; Yoo, L. Penetration of Anti-Infective Agents into Pulmonary Epithelial Lining Fluid. Clinical Pharmacokinetics 2011 50:10 2012, 50 (10), 637-664. DOI: 10.2165/11594090-000000000-00000.
Himstedt, A.; Braun, C.; Wicha, S. G.; Borghardt, J. M. Understanding the Suitability of Established Antibiotics for Oral Inhalation from a Pharmacokinetic Perspective: An Integrated Model-Based Investigation Based on Rifampicin, Ciprofloxacin and Tigecycline in Vivo Data. J Antimicrob Chemother 2022, 77 (11), 2922-2932.
Betts, J. W.; Phee, L. M.; Woodford, N.; Wareham, D. W. Activity of Colistin in Combination with Tigecycline or Rifampicin against Multidrug-Resistant Stenotrophomonas maltophilia. European Journal of Clinical Microbiology and Infectious Diseases 2014, 33 (9), 1565-1572.
C, Y.; W, S.; L, H.; Y, L.; YH, Z.; Y, Y.; Y, Z.; JB, L. Effects of Tigecycline Combined with Azithromycin Against Biofilms of Multidrug-Resistant Stenotrophomonas maltophilia Isolates from a Patient in China. Infect Drug Resist 2021, Volume 14, 775-786.

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A composition, comprising:
a micronized tigecycline having a Dv90 mean particle size of less than 7 μm, wherein the composition is a powder, wherein the composition is carrier free, and wherein the composition is free of any stabilizing excipients.

2. The composition of claim 1, wherein the micronized tigecycline has a Dv90 mean particle size of less than 6 μm.

3. The composition of claim 1, wherein the micronized tigecycline has a Dv90 mean particle size of less than 5 μm.

4. The composition of claim 1, wherein the micronized tigecycline is deliverable by aerosolization, a dry powder inhaler, a metered dose inhaler, an insufflator, or a dry powder nasal spray.

5. The composition of claim 1, wherein a fine particle fraction of the micronized tigecycline is at least 70%.

6. The composition of claim 1, wherein the mean particle size of the micronized tigecycline in the composition is from 0.5 μm to 5 μm.

7. The composition of claim 1, wherein the composition is a storage stable composition.

8. The composition of claim 7, wherein the composition is stable at a temperature of at least 25° C. for at least one month.

9. The composition of claim 7, wherein the composition is stable at a temperature of at least 25° C. and a relative humidity of 60% for at least one month.

10. The composition of claim 7, wherein the composition is stable at a temperature of at least 25° C. for at least six months.

11. The composition of claim 7, wherein the composition is stable at a temperature of at least 25° C. and a relative humidity of 60% for at least six months.

12. The composition of claim 7, wherein the composition is stable at a temperature of at least 40° C. for at least one month.

13. The composition of claim 7, wherein the composition is stable at a temperature of at least 40° C. and a relative humidity of 75% for at least one month.

14. The composition of claim 7, wherein the composition is stable at a temperature of at least 40° C. for at least six months.

15. The composition of claim 7, wherein the composition is stable at a temperature of at least 40° C. and a relative humidity of 75% for at least six months.

16. The composition of claim 1, wherein the micronized tigecycline exhibits a specific surface area of at least 23 m²/g.

17. The composition of claim 1, wherein a fine particle dose of the micronized tigecycline is at least 9.2 mg.

18. The composition of claim 1, wherein the mass median aerodynamic diameter of the micronized tigecycline is less than 2.1 μm.

19. The composition of claim 1, wherein the micronized tigecycline has a Dv50 mean particle size of less than 3 μm.

20. (canceled)

21. (canceled)

22. A method of treating a pulmonary infection in a subject, comprising:
administering to a subject the composition of claim 1.

23.-41. (canceled)
